(19) United States
(12) Patent Application Publication
(10) Pub. No.: US 2023/0277230 A1
(43) Pub. Date: Sep. 7, 2023

(54) Graft fixation screw with tangs

(71) Applicant: Orthopedic Designs North America, Inc., Tampa, FL (US)

(72) Inventors: Lance Fagan, Bartow, FL (US); Bradley Young, Atlanta, GA (US)

(21) Appl. No.: 17/934,791

(22) Filed: Sep. 23, 2022

Related U.S. Application Data
(63) Continuation-in-part of application No. 63/315,136, filed on Mar. 1, 2022.

(51) Int. Cl. A61B 17/86; A61F 2/08

(52) U.S. Cl. CPC A61B 17/8645; A61F 2/0811; A61F 2002/0817; A61B 2017/8655

(57) ABSTRACT

The graft fixation device with tangs combines a fixation screw body with tangs that extend from an internal position to an external position, the tangs piercing and gripping the bone and the graft. The use of tangs overcomes the prior art limitations of relying exclusively on compression, which increased the risk of failure, including failure modes such as the graft sliding out of the hole.

### PRIORITY

This application claims priority to U.S. provisional application No. 63/315,136 titled Fixation Screw with Deployable Tangs, filed Mar. 1, 2022.

### FIELD

This invention relates to the field of graft fixation and more particularly to a device for fixation of a graft within a hole in a bone.

### BACKGROUND

Ligament and tendon repair often require fixation of a graft, tendon, suture, or ligament to a bone.

Prior art methodologies of fixation include a screw that compresses the graft against the wall of the hole within the bone.

This methodology of fixation is imperfect because it requires compression of the graft between the screw and the hole.

What is needed is a device for fixation of grafts within a bone that does not rely on matching the screw and hole diameter, and that does not depend upon compression as the exclusive means of fixation.

### SUMMARY

The graft fixation device with tangs combines a fixation screw body with tangs that extend from an internal position to an external position, the tangs piercing and gripping the bone and the graft.

The use of tangs overcomes the prior art limitations of relying exclusively on compression, which increased the risk of failure, including failure modes such as the graft sliding out of the hole. The reliance on compression also created difficulties during surgery. For example, if the screw did not create adequate compression of the graft within the hole, a larger diameter hole was required for a larger screw.

In some situations, a larger hole cannot be drilled due to the constraints of the bone, or the use of a larger screw is inappropriate. In such situations, the graft fixation screw with tangs can be used to fix the ligament inside the hole even in the absence of adequate compression fixation. This is because the graft fixation screw with tangs relies on piercing tangs to grip the graft or suture, and not merely on compression of the graft.

Each tang includes a piercing tip that broadens to an expanded bearing surface. During extension of the tangs, the tip of a tang pierces the graft, then passes through the bone. As a result, the graft is pinned in place against the bone. This prevents the graft from sliding out of the hole, and prevents rotation of the graft fixation device within the hole.

In the preferred embodiment, the body of the graft fixation screw is fully threaded with the exception of a zero-lead thread along the top of the screw that acts as a stop or plug.

The motion of the tangs, and their associated threaded collars, is controlled by a threaded actuator.

The threaded actuator preferably includes two sections of threads, one for each collar. The two sections of threads are preferably of opposite handedness.

Handedness is the direction of linear motion caused by rotation. Viewed from above, a right-handed thread will move away when rotated clockwise, and toward when rotated counterclockwise. A left-handed thread is the opposite, moving away when rotated counterclockwise and toward when rotated clockwise.

By having two sections of threads, the two sections having opposite handedness, rotation of the threaded actuator causes the associated collars to have opposite linear motion associated with the same rotation, therefore moving toward and away from each other during rotation.

The graft fixation screw with tangs can be used to fix various types of materials to bone, including grafts, tendons, sutures, and synthetic mesh, as well as other types of artificial soft tissue.

While the primary embodiment of the graft fixation screw with tangs is intended for use in human surgery, use in animal surgery is also anticipated. Furthermore, non-surgical applications, such as fixation of two or more layers of material that share a common hole, or fixation of any linear element inside a hole drilled in, for example wood or concrete, are also anticipated.

The disclosed primary embodiment includes two tangs and a single external body thread. Variations of the primary embodiment are anticipated, including changes in diameter, quantity of threads, pitch of threads, number of tangs, shape of tangs, and position of tangs.

### BRIEF DESCRIPTION OF THE DRAWINGS

The invention can be best understood by those having ordinary skill in the art by reference to the following detailed description when considered in conjunction with the accompanying drawings in which:

FIG. 1A illustrates a first isometric view of the graft fixation device with tangs retracted.

FIG. 1B illustrates a first isometric view of the graft fixation device with tangs extended.

FIG. 2A illustrates a cross-sectional view of the graft fixation device with tangs retracted.

FIG. 2B illustrates a cross-sectional view of the graft fixation device with tangs extended.

FIG. 3A illustrates a bone with hole for installation of the graft fixation device.

FIG. 3B illustrates a bone with hole with the graft fixation device installed.

FIG. 3C illustrates a bone with hole with the graft fixation device installed and tangs extended.

FIG. 4A illustrates a top view of the graft fixation device with tangs retracted.

FIG. 4B illustrates a top view of the graft fixation device with tangs extended.

FIG. 5A illustrates a side view of the graft fixation device with tangs retracted.

FIG. 5B illustrates a side view of the graft fixation device with tangs extended.

FIG. 6A illustrates a bottom view of the graft fixation device with tangs retracted.

FIG. 6B illustrates a bottom view of the graft fixation device with tangs extended.

FIG. 7A illustrates a view of the tangs and actuator of the graft fixation device with tangs retracted.

FIG. 7B illustrates a view of the tangs and actuator of the graft fixation device with tangs extended.

FIG. 8 illustrates a cross-sectional view of the body of the graft fixation device.

FIG. 9 illustrates an exploded view of the graft fixation device.

FIG. 10 illustrates a view of the tangs of the graft fixation device.

FIG. 11 illustrates a view of the installation tool interfacing with the graft fixation device.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

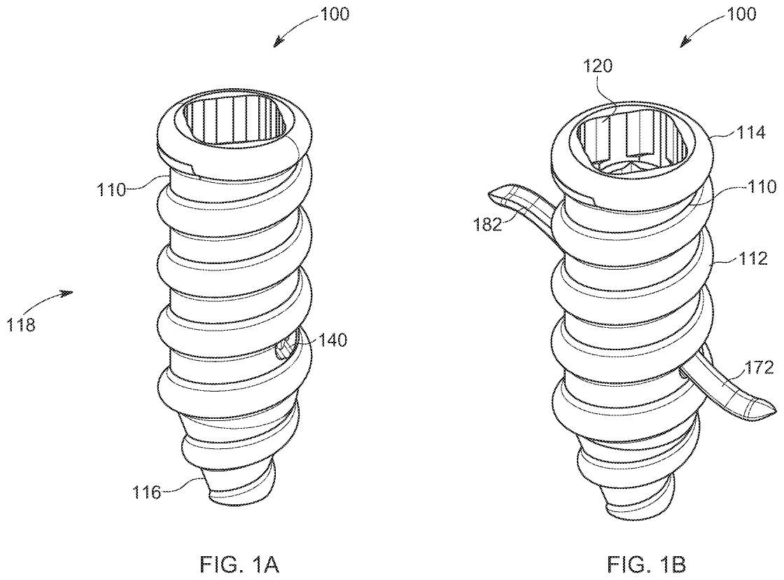

Referring to FIGS. 1A and 1B, a first isometric view of the graft fixation device with tangs is shown.

The graft fixation screw with tangs 100 includes a body no with external thread 112 and optional zero-lead top thread 114. To aid in insertion, the body 110 narrows at the insertion end, or lower end, creating a tapered tip 116, increasing in diameter to a consistent-diameter central section 118 that continues through to the upper end.

A body tool interface 120 allows the surgeon's tool to control rotation of the body no.

A first set of tangs 172 and a second set of tangs 182 are shown, each tang 172/182 exiting the body no through a tang deflection passage 140.

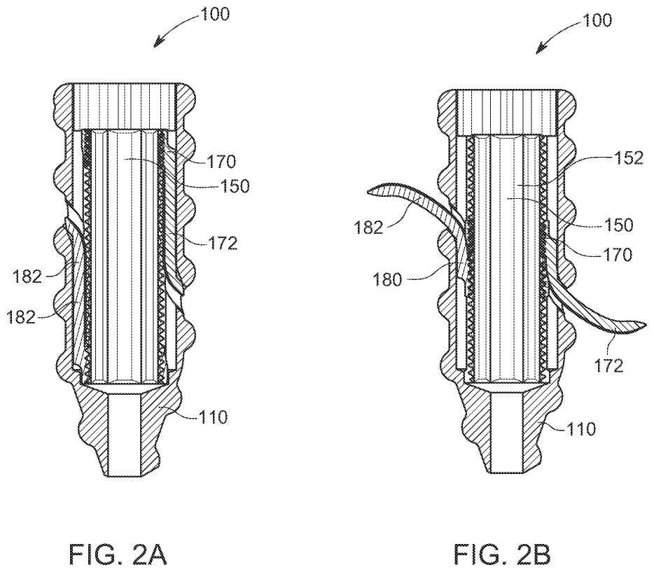

Referring to FIGS. 2A and 2B, a cross-sectional view of the graft fixation device with tangs is shown.

As the threaded actuator 150 rotates, the first collar 170 moves toward the second collar 180, pushing the first set of tangs 172 and the second set of tangs 182 through the body 110. Stated differently, the first collar 170 and the second collar 180 start from a separated position and move to an adjacent position. It is during this transition between positions that the first set of tags 172 and the second set of tags 182 are extended.

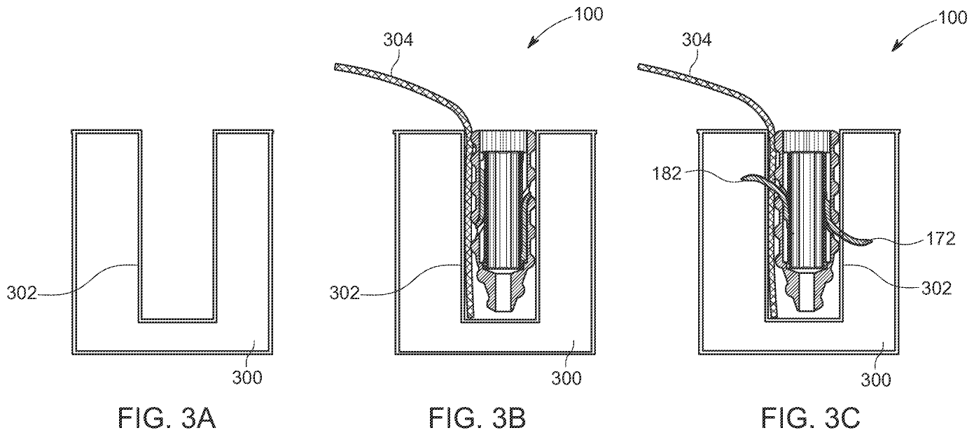

Referring to FIGS. 3A, 3B, and 3C installation of the graft fixation device installed is shown.

FIGS. 3A-C depict the graft fixation device used in a tenodesis procedure, which is the fixation of a graft into a socket with a base to prevent the graft from sliding retrograde out of the hole.

The graft fixation device can also be used to accomplish interference fixation, or fixation of a graft in a tunnel. To fix a graft within a tunnel, the graft enters the tunnel on a first end and exits on a second end. The graft fixation device is placed within the tunnel, preventing the graft from sliding antegrade or retrograde.

An example bone 300 is shown with hole 302. For fixation, a graft 304 is placed within the hole 302. The graft fixation screw with tangs 100 is threaded into the hole 302, preferably compressing the graft 304 against the bone 300, although compression is not required.

Tangs 172/182 are then extended, piercing the graft 304 and pushing into the bone 300, fixing the graft 304 with respect to the bone 300.

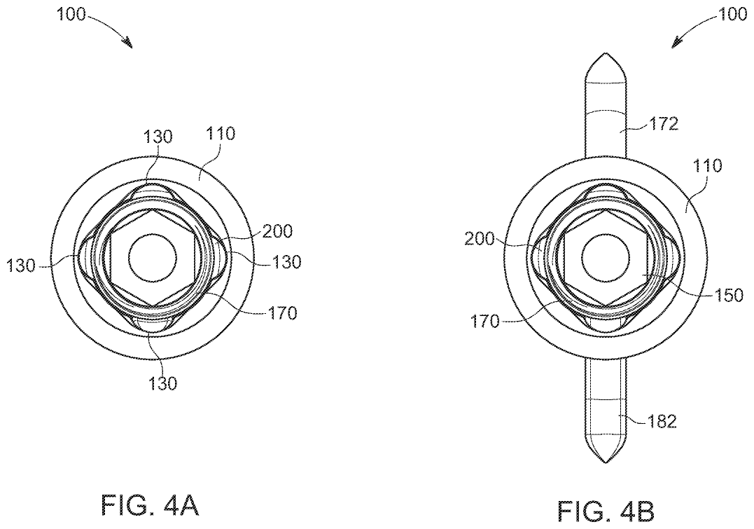

Referring to FIGS. 4A and 4B, a top view of the graft fixation device with tangs is shown.

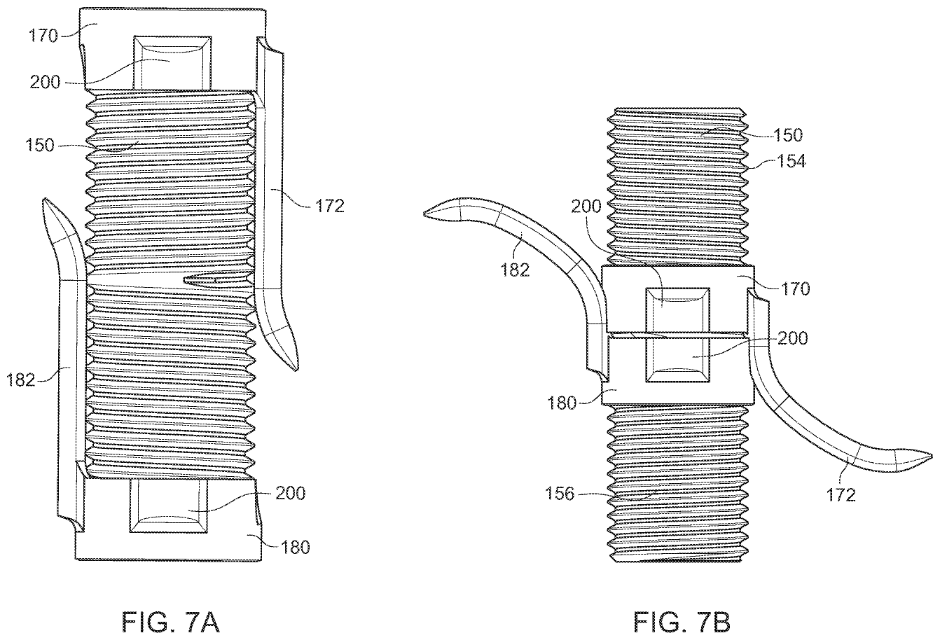

The body no includes one or more linear channels 130 recessed into the body 110. The first collar 170 and the second collar 180 (see FIGS. 7A and 7B) include anti-rotation projections 200 that slide within the linear channels 130, preventing rotation of the collars 170/180 while the threaded actuator 150 rotates. The result is that the collars 170/180 are only permitted to slide, and prohibited from rotation—resisting rotation by being indexed at a fixed angular position with respect to the body 110.

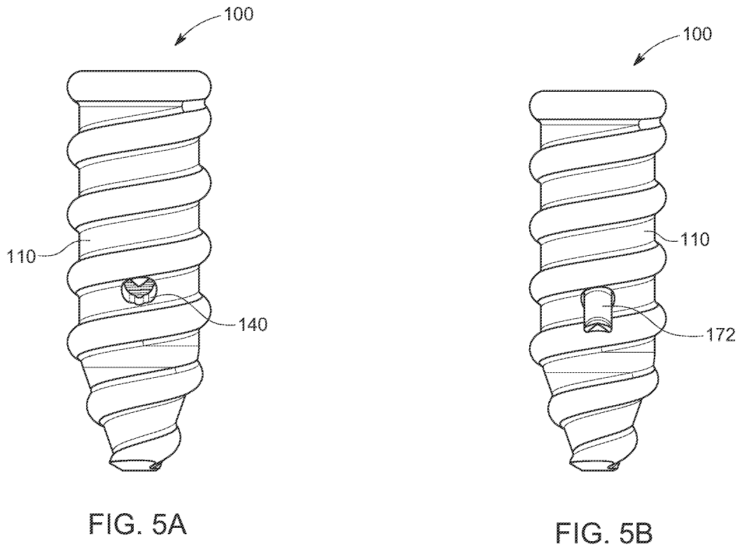

Referring to FIGS. 5A and 5B, a side view of the graft fixation device with tangs is shown.

The tip of the first set of tangs 172 is partially visible through the tang deflection passage 140 opening through the body 110.

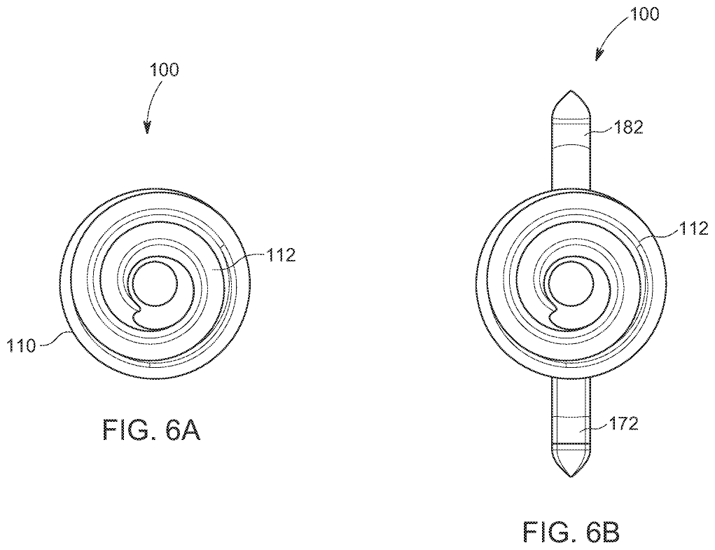

Referring to FIGS. 6A and 6B, a bottom view of the graft fixation device with tangs is shown.

The external thread 112 is shown around the body 110 with the first set of tangs 172 and the second set of tangs 182 protruding beyond the threads after extension. Each set of tang 172/182 can be a single tang, or two or more tangs.

Referring to FIGS. 7A and 7B, a view of the tangs and actuator of the graft fixation device with tangs is shown.

The first collar 170 with first set of tangs 172 is shown, with the second collar 180 including a second set of tangs 182.

The position of the first collar 170 is controlled by the first thread set 154 of the threaded actuator 150. The position of the second collar 180 is controlled by the second thread set 156 of the threaded actuator 150.

In the preferred embodiment, the first thread set 154 and the second thread set 156 are of opposite handedness—one being a left hand thread, the other being a right hand thread. The result is that rotation of the threaded actuator 150 causes opposite linear motion of the first collar 170 and the second collar 180, causing the collars to move toward each other—from a separated position to an adjacent position.

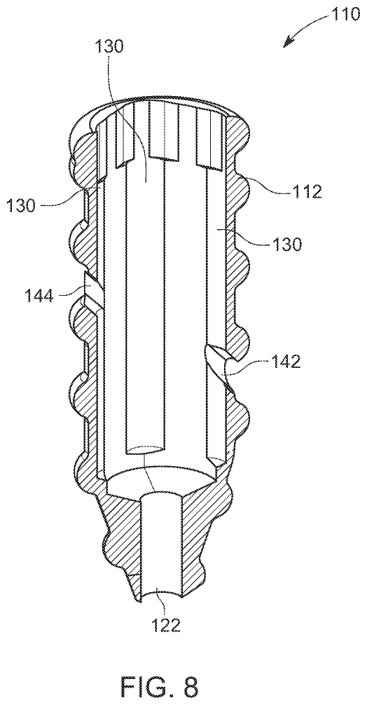

Each collar 170/180 includes one or more anti-rotation projections 200 that interface with the linear channels 130 of the body 110 (see FIG. 8).

Referring to FIG. 8, a cross-sectional view of the body of the graft fixation device is shown.

The body no includes external thread 112 and internal linear channels 130. To guide the tangs 172/182 during extension, the body 110 includes a downward deflection ramp 142 and an upward deflection ramp 144.

Also shown is through-hole 122, allowing the graft fixation screw with tangs 100 to be slid over a wire or guide rod.

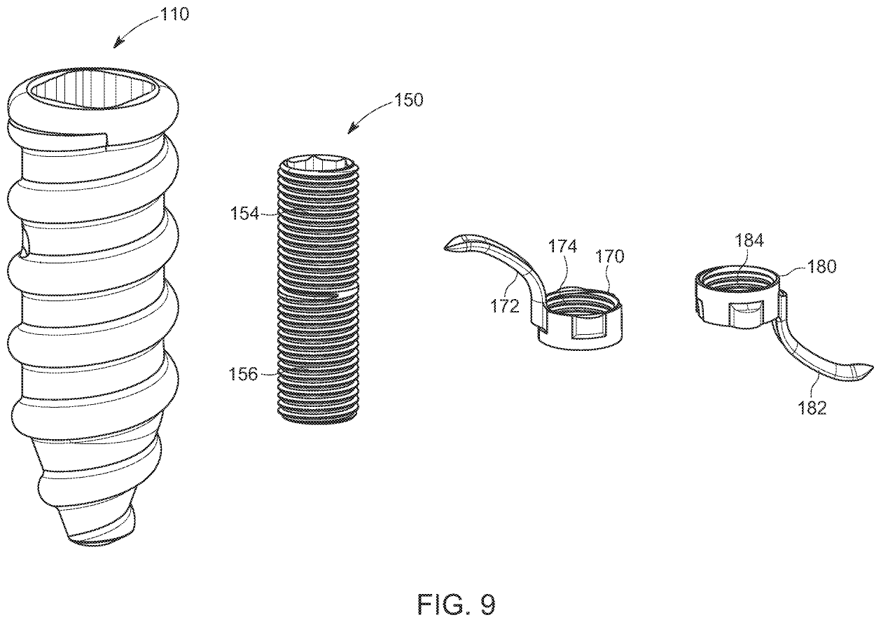

Referring to FIG. 9, an exploded view of the graft fixation device is shown.

Shown is body 110, threaded actuator 150 with first thread set 154 and second thread set 156, and first collar 170 with first set of tangs 172 and first set of internal threads 174. Also shown is second collar 180 with second set of tangs 182 and second set of internal threads 184.

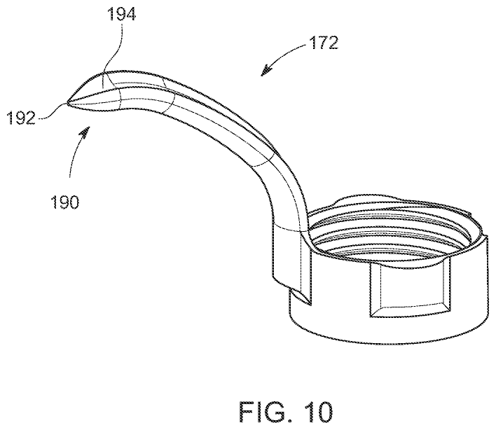

Referring to FIG. 10, a view of the tangs of the graft fixation device is shown.

Each set of tangs, shown here as first set of tangs 172, includes a pre-curved tip 190. The pre-curved tip 190 helps the first set of tangs 172 to catch the tang deflection passage 140 (see FIG. 8) as extension begins.

The piercing tip 192 allows the tang 172 to push through and separate, rather than cut, the graft 304 (see FIG. 3C). As the tang 172 continues to push through the graft 304, the broadening bearing surface 194 of the tang 172 spreads the load to discourage the graft 304 from ripping or tearing.

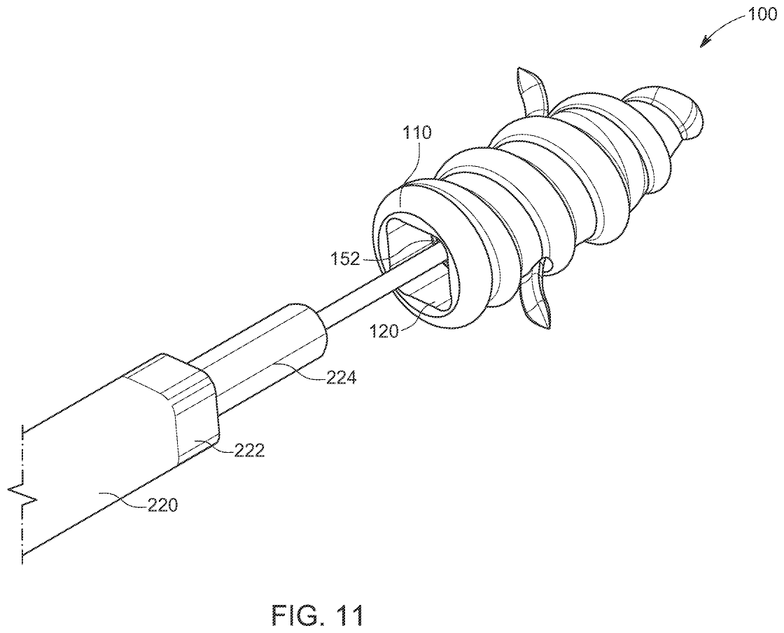

Referring to FIG. 11, a view of the tool interfacing with the graft fixation device is shown.

To allow the surgeon to manipulate the graft fixation screw with tangs 100, the tool shaft 220 includes a body interface 222 that rotationally locks to the body tool interface 120.

A secondary threaded actuator interface 224 rotationally locks to the actuator tool interface 152, allowing the surgeon to rotate the threaded actuator 150 (see FIG. 2B) while holding the body 110 stationary.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A device for fixation of a graft within a hole of a bone, the device comprising:
a body;
the body including an external surface, internal surface, an upper end, and a lower end;
the body including an external thread from the upper end to the lower end;
a threaded actuator;
a first tang affixed to a first collar;
the first collar interfacing with the threaded actuator;
a second tang affixed to a second collar;
the second collar interfacing with the threaded actuator;
rotation of the threaded actuator causing the first collar and the second collar to move from a separated position to an adjacent position, causing the first tang and the second tang to protrude through the body;
whereby of the external thread, the first tang, and the second tang cooperate to secure a graft within the hole of the bone.

2. The device for fixation of a graft within a hole of a bone of claim 1, wherein the first tang includes a piercing tip;
the piercing tip pushing through and separating the graft, rather than cutting the graft, acting to pin the graft to the bone.

3. The device for fixation of a graft within a hole of a bone of claim 1, the body further comprising a linear channel;
the linear channel along the internal surface of the body;
the first collar sliding along the linear channel as the first collar moves from the separated position to the adjacent position;
the linear channel preventing rotation of the first collar.

4. The device for fixation of a graft within a hole of a bone of claim 1, further comprising:
an anti-rotation projection;
the anti-rotation projection extending from the first collar;
a linear channel;
the linear channel recessed into the internal surface of the body;
the anti-rotation projection sliding along the linear channel as the first collar moves with respect to the body;
wherein interaction of the anti-rotation projection and the linear channel prevents rotation of the first collar with respect to the body.

5. The device for fixation of a graft within a hole of a bone of claim 4, wherein the first tang includes a piercing tip;
the piercing tip pushing through and separating the graft, rather than cutting the graft, acting to pin the graft against the bone.

6. The device for fixation of a graft within a hole of a bone of claim 1, wherein the threaded actuator includes a first thread set and a second thread set;
the first collar interfacing with the first thread set;
the second collar interfacing with the second thread set;
the first thread set and the second thread set having opposite handedness, whereby rotation of the threaded actuator causes motion of the first collar toward the second collar.

7. A graft fixation device for insertion into a hole in a bone to prevent withdrawal of a graft with respect to the bone, the graft fixation device comprising:
a body, the body including an internal surface;
a first collar within the body;
the first collar permitted to slide with respect to the body;
the first collar prohibited from rotating with respect to the body;
a threaded actuator;
the threaded actuator interfacing with the first collar;
rotation of the threaded actuator causing linear motion of the first collar;
a first set of tangs;
the first set of tangs interfacing with the first collar;
linear motion of the first collar causing extension of the first set of tangs through the body;
whereby the body is placed into the hole in the bone, with the graft between the body and a wall of the hole, the first set of tangs extending and piercing the graft, pinning the graft against the bone.

8. The graft fixation device of claim 7, wherein a tang of the first set of tangs includes a piercing tip;
the piercing tip pushing through and separating the graft, rather than cutting the graft, acting to pin the graft against the bone.

9. The graft fixation device of claim 7, the body further comprising a linear channel;
the linear channel along the internal surface of the body;
the first collar sliding along the linear channel;
the linear channel preventing rotation of the first collar.

10. The graft fixation device of claim 7, further comprising:
an anti-rotation projection;
the anti-rotation projection extending from the first collar;
a linear channel;
the linear channel recessed into the internal surface of the body;
the anti-rotation projection sliding along the linear channel as the first collar moves with respect to the body;
wherein interaction of the anti-rotation projection and the linear channel prevents rotation of the first collar with respect to the body.

11. The graft fixation device of claim 10, wherein a tang of the first set of tangs includes a piercing tip;
the piercing tip pushing through and separating the graft, rather than cutting the graft, acting to pin the graft against the bone.

12. The graft fixation device of claim 7, wherein the threaded actuator includes a first thread set and a second thread set;
the first collar interfacing with the first thread set;
a second collar interfacing with the second thread set;
the first thread set and the second thread set have opposite handedness, whereby rotation of the threaded actuator causing motion of the first collar toward the second collar.

13. A device to fix a ligament within a bone comprising:
a fully threaded body surrounding a threaded actuator;
the threaded actuator including a first thread set and a second thread set;
a first collar with a first set of internal threads that interact with the first thread set of the threaded actuator;
movement of the first collar with respect to the fully threaded body causing extension and retraction of a first set of tangs;
a second collar with a second set of internal threads that interact with the second thread set of the threaded actuator;
movement of the second collar with respect to the fully threaded body causing extension and retraction of a second set of tangs;
whereby a surgeon rotates the fully threaded body into a hole within the bone, then rotates the threaded actuator to cause extension of the first set of tangs and extension of the second set of tangs into the bone.

14. The device to fix a ligament within a bone of claim 13, wherein a first tang of the first set of tangs includes a piercing tip;
the piercing tip pushing through and separating the ligament, rather than cutting the ligament, acting to pin the ligament against the bone.

15. The device to fix a ligament within a bone of claim 13, the fully threaded body further comprising a linear channel;
the linear channel along an internal surface of the fully threaded body;
the first collar sliding along the linear channel as the first collar moves;
the linear channel preventing rotation of the first collar.

16. The device to fix a ligament within a bone of claim 13, further comprising:
an anti-rotation projection;
the anti-rotation projection extending from the first collar;
a linear channel;
the linear channel recessed into an internal surface of the fully threaded body;
the anti-rotation projection sliding along the linear channel as the first collar moves;
wherein interaction of the anti-rotation projection and the linear channel prevents rotation of the first collar with respect to the fully threaded body.

17. The device to fix a ligament within a bone of claim 16, wherein a first tang of the first set of tangs includes a piercing tip;
the piercing tip pushing through and separating the ligament, rather than cutting the ligament, acting to pin the ligament against the bone.

18. The device to fix a ligament within a bone of claim 13, wherein the threaded actuator includes a first thread set and a second thread set;
the first collar interfacing with the first thread set;
the second collar interfacing with the second thread set;
the first thread set and the second thread set having opposite handedness, rotation of the threaded actuator causing motion of the first collar toward the second collar.
